(19) United States
(12) Patent Application Publication
(10) Pub. No.: US 2020/0111564 A1
(43) Pub. Date: Apr. 9, 2020

(54) METHOD AND APPARATUS FOR AUTOMATING OPERATING ROOMS WITH HANDHELD ELECTRONIC DEVICES

(71) Applicant: ReadyOR, LLC, Wintersville, OH (US)

(72) Inventor: Nicholas P. Mastros, Wintersville, OH (US)

(21) Appl. No.: 16/395,811

(22) Filed: Apr. 26, 2019

Related U.S. Application Data
(60) Provisional application No. 62/741,597, filed on Oct. 5, 2018.

(51) Int. Cl. G16H 40/20; G16H 40/63; G16H 30/20; G16H 70/20; G06F 3/0482; G06Q 10/08

(52) U.S. Cl. CPC G16H 40/20; G16H 40/63; G06Q 10/0875; G16H 70/20; G06F 3/0482; G16H 30/20

(57) ABSTRACT

A system for ensuring proper set-up of an operating room and proper use of surgical tools during surgery includes a handheld electronic device, an electronic database, wherein the electronic database either resides on the handheld electronic database or the handheld electronic database has remote access to the electronic database, an electronic list of physicians stored in the electronic database, at least one electronic image of a preferred operating room set-up for each of the physicians for each of the physician's surgical procedures, wherein the at least one electronic image of the preferred operating room set up shows each piece of surgical equipment for each surgical procedure and a preferred location for each piece of surgical equipment for each surgical procedure, wherein the at least one electronic image is stored in the electronic database.

This application claims priority to a provisional patent application, entitled Method and Apparatus for Enhancing Operating Room Efficiency, having Ser. No. 62/741,597, filed Oct. 5, 2018. The present teachings generally relate to methods and apparatuses for surgery, and more particularly to automating operating rooms with handheld electronic devices, and even more particularly to utilizing handheld electronic devices operating software to ensure proper set-up of the operating room, and proper utilization of surgical tools during surgery.

### BACKGROUND

### A. Field

### B. Background

There are over 5,000 surgical center and/or hospitals and hospitals in the United States, where millions of surgeries are performed every year. In the United States, there are over 18,000 surgeons performing these millions of surgeries.

Every year, thousands of mistakes are made during surgeries, some preventable, some not. The surgical procedure involves, hospitals, patients, nurses, surgeons, anesthesiologists, insurance companies, as well as host of necessary equipment and material. Improving the efficiency of the surgeons and surgical procedures could cut down on both the cost and the mistakes made during surgery.

Each surgeon within a surgical center and/or hospital typically has a preference for which equipment to use for a particular surgery, as well as the set up of the operating room prior to commencement of surgery. These preferences are typically kept in a preference book or note cards, and are consulted prior to each surgery.

### II. SUMMARY

In accordance with one aspect of the present teachings, a handheld electronic device utilizing a software app is used to efficiently store and update surgeon's operating room preferences.

In accordance with one aspect of the present teachings, a handheld electronic device utilizing a software app can track all of the different time components of pre-op and the actual surgery.

In accordance with one aspect of the present teachings, a handheld electronic device utilizing a software app contains pictures of the surgeon's preferred room set-up.

In accordance with one aspect of the present teachings, a system for ensuring proper set-up of an operating room and proper use of surgical tools during surgery includes a handheld electronic device, an electronic database, wherein the electronic database either resides on the handheld electronic database or the handheld electronic database has remote access to the electronic database, an electronic list of physicians stored in the electronic database, at least one electronic image of a preferred operating room set-up for each of the physicians for each of the physician's surgical procedures, wherein the at least one electronic image of the preferred operating room set up shows each piece of surgical equipment for each surgical procedure and a preferred location for each piece of surgical equipment for each surgical procedure, wherein the at least one electronic image is stored in the electronic database, an electronically selectable list of billable supplies, wherein the electronically selectable list of billable supplies is stored on the electronic database, wherein the electronic list of billable supplies is automatically transmittable to an associated patient and/or an associated insurance company, at least one instructional video, wherein the at least one instructional video contains instructions on use of the surgical equipment, wherein the instructional video contains instructions on how to trouble shoot the surgical equipment, wherein the at least one instructional video is stored on the electronic database, and a time tracker for tracking stages of an operation, wherein the time tracker is stored on the electronic database.

In accordance with one aspect of the present teachings, the system further comprises electronic circulator notes.

In accordance with one aspect of the present teachings, the time tracker comprises entry time when the associated patient enter the operating room, surgery starting time, surgery ending time, and time when the associated patient is moved to a recovery room.

In accordance with one aspect of the present teachings, a system for ensuring proper set-up of an operating room and proper use of surgical tools during surgery includes a handheld electronic device, an electronic database, wherein the electronic database either resides on the handheld electronic database or the handheld electronic database has remote access to the electronic database, an electronic list of physicians stored in the electronic database, at least one electronic image of a preferred operating room set-up for each of the physicians for each of the physician's surgical procedures, wherein the at least one electronic image of the preferred operating room set up shows each piece of surgical equipment for each surgical procedure and a preferred location for each piece of surgical equipment for each surgical procedure, wherein the at least one electronic image is stored in the electronic database.

In accordance with one aspect of the present teachings, the system further comprises an electronically selectable list of billable supplies, wherein the electronically selectable list of billable supplies is stored on the electronic database, wherein the electronic list of billable supplies is automatically transmittable to an associated patient and/or an associated insurance company.

In accordance with one aspect of the present teachings, the system further comprises at least one instructional video, wherein the at least one instructional video contains instructions on use of the surgical equipment, wherein the instructional video contains instructions on how to trouble shoot the surgical equipment, wherein the at least one instructional video is stored on the electronic database.

In accordance with one aspect of the present teachings, wherein the system further comprises a time tracker for tracking stages of a surgical procedure, wherein the time tracker is stored on the electronic database.

In accordance with one aspect of the present teachings, the system further comprises multiple participant real time video functionality, wherein the multiple participant real time video functionality is stored on the electronic database.

In accordance with one aspect of the present teachings, the system further comprises electronic circulator notes.

In accordance with one aspect of the present teachings, the time tracker comprises entry time when the associated patient enter the operating room, surgery starting time, surgery ending time, and time when the associated patient is moved to a recovery room.

In accordance with one aspect of the present teachings, a method for ensuring proper set-up of an operating room and proper use of surgical tools during surgery, comprises the steps of providing a handheld electronic device, providing an electronic database, wherein the electronic database either resides on the handheld electronic database or the handheld electronic database has remote access to the electronic database, storing an electronic list of physicians on the electronic database, and storing at least one electronic image of a preferred operating room set-up for each of the physicians for each of the physician's surgical procedures, wherein the at least one electronic image of the preferred operating room set up shows each piece of surgical equipment for each surgical procedure and a preferred location for each piece of surgical equipment for each surgical procedure, wherein the at least one electronic image is stored in the electronic database.

In accordance with one aspect of the present teachings, the method further comprises the steps of electronically selecting a list of billable supplies, wherein the electronically selectable list of billable supplies is stored on the electronic database, creating an invoice based on billable supplies used during the surgical procedure, and automatically transmitting the invoice to an associated patient and/or an associated insurance company.

In accordance with one aspect of the present teachings, the method further comprises the steps of creating at least one instructional video, wherein the at least one instructional video contains instructions on use of the surgical equipment, wherein the instructional video contains instructions on how to trouble shoot the surgical equipment and storing the at least one instructional video on the electronic database.

In accordance with one aspect of the present teachings, the method further comprises the steps of tracking time for stages of the surgical procedure and storing tracked time on the electronic database.

In accordance with one aspect of the present teachings, the method further comprises the step of providing multiple participant real time video functionality, wherein the multiple participant real time video functionality is stored on the electronic database.

In accordance with one aspect of the present teachings, the method further comprises the step of providing electronic circulator notes.

In accordance with one aspect of the present teachings, the method further comprises the steps of tracking an entry time when the associated patient enter the operating room, tracking surgery starting time, tracking surgery ending time, and tracking time when the associated patient is moved to a recovery room.

Other benefits and advantages will become apparent to those skilled in the art to which it pertains upon reading and understanding of the following detailed specification.

### III. BRIEF DESCRIPTION OF THE DRAWINGS

The present teachings are described hereinafter with reference to the accompanying drawings.

FIG. 1 shows a plan view of a screen shot of the software app on the handheld electronic device;

FIG. 2 shows a plan view of a screen shot of the software app on the handheld electronic device;

FIG. 3 shows a plan view of a screen shot of the software app on the handheld electronic device;

FIG. 4 shows a plan view of a screen shot of the software app on the handheld electronic device;

FIG. 5 shows a plan view of a screen shot of the software app on the handheld electronic device;

FIG. 6 shows a plan view of a screen shot of the software app on the handheld electronic device;

FIG. 7 shows a plan view of a screen shot of the software app on the handheld electronic device; and

FIG. 8 shows a plan view of a screen shot of the software app on the handheld electronic device.

### IV. DETAILED DESCRIPTION

The set-up of an operating room for surgery is dependent upon not only the surgical center and/or hospital, and the type of surgery being performed, but also by the preference of the surgeon performing the surgery. Each surgeon has a preference as to which pieces of equipment they prefer, and where they would like the equipment to be placed in the operating room. Currently, these preferences are kept in laminated preference books or note cards, which the surgical staff must refer to and keep up to date for each surgeon.

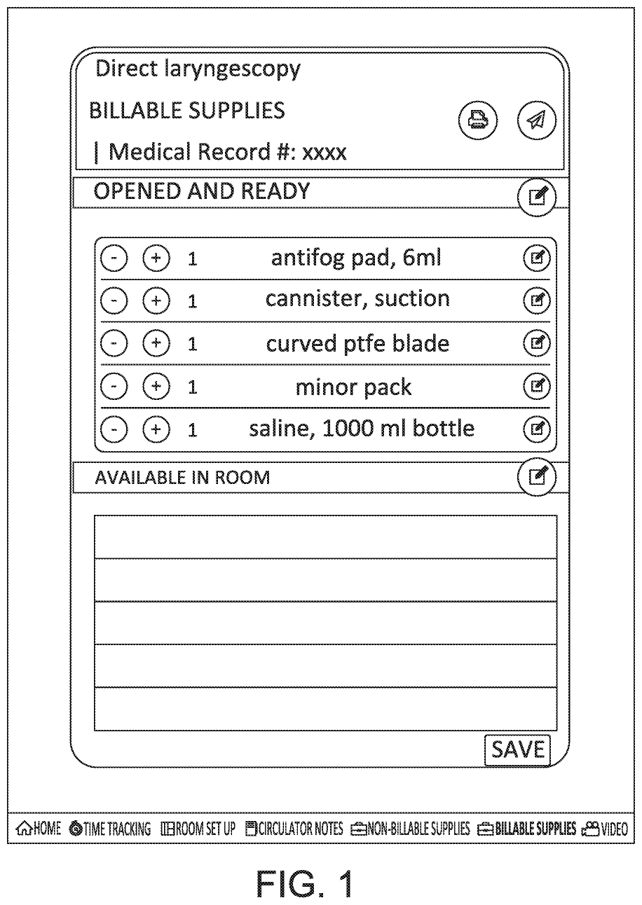

In one aspect of the present teachings, a system for automating operating rooms uses a handheld electronic device utilizing a software provides the necessary information formerly kept in preference books or note cards, along with additional useful information. With respect to FIGS. 1-8, the software, which in one aspect of the present teachings is a mobile app, can store preference information for all of the surgeons in a particular surgical center and/or hospital, so that the surgical staff can ensure that the operating room is set up as preferred by the surgeon. FIG. 1 shows a portion of the software related to billable supplies. The system allows the user to designate which billable supplies are to be included in the room, and the number. This creates a list of all the items that can be billed to the patient and/or insurance company. Once the billable supplies list is set up, this information can be automatically sent to the internal billing office of the surgical center and/or hospital. It is also to be understood that this information could be automatically transmitted to a patient and/or an insurance company in the form of an invoice. This portion of the software will show the billable supplies that are in the room, and can be listed as opened and ready for use. Whether or not the supplies have been opened can be directly linked to the transmission of data to the internal billing office and/or the insurance company.

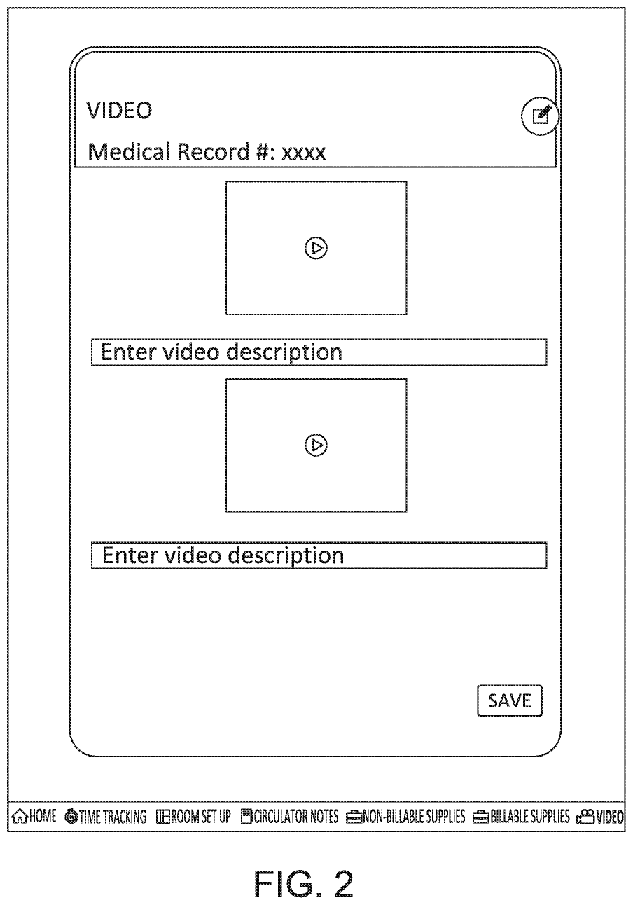

With respect to FIG. 2, videos can be stored on an electronic database, which either resides on the electronic handheld device, or can be remotely accessed by the handheld device, such as through the cloud. These videos can provide instructions on the use of particular surgical device. Currently, a medical equipment representative is physically present in the operating room during surgery, in case the medical staff have questions about the use and/or malfunction of a piece of equipment. The videos stored in the software can provide all the details about how to use the medical equipment, and how to trouble shoot it should problems arise. This will eliminate the need for medical equipment representatives to be in the operating room. The medical equipment company could supply the videos, or the videos could be created in the surgical center and/or hospital, but the videos will be available prior to commencement of the surgery.

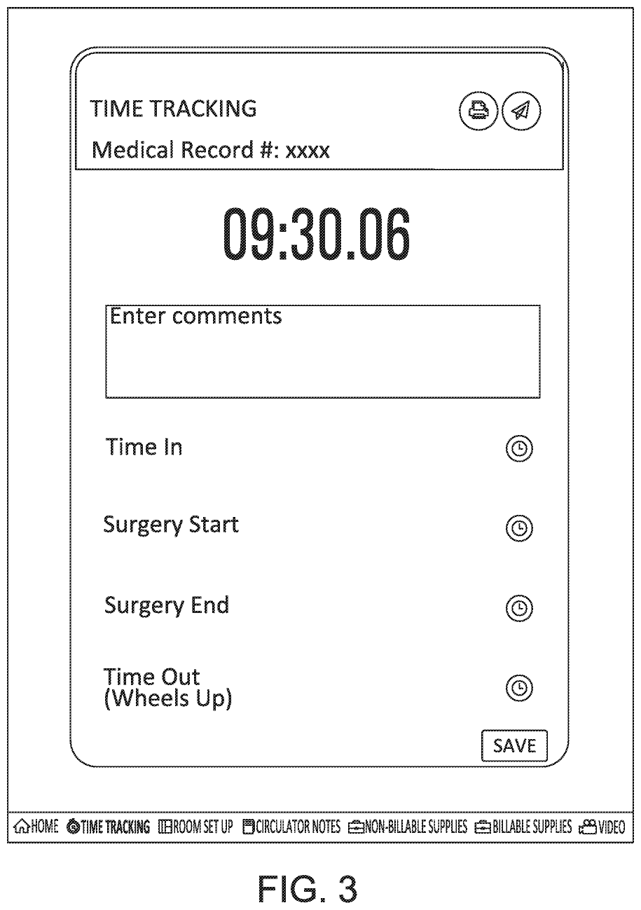

With respect to FIG. 3, the system contains a time tracking functionality. The time tracker can keep track of the time the patient enters the operating room, the time the surgery starts, the time the surgery ends, and when the patient is moved from the operating room to recovery. This time can be tracked both by operating room, as well as a particular surgeon. This allows the surgical center and/or hospital to determine if the cause of delays is the staff or the surgeon, or some combination of both.

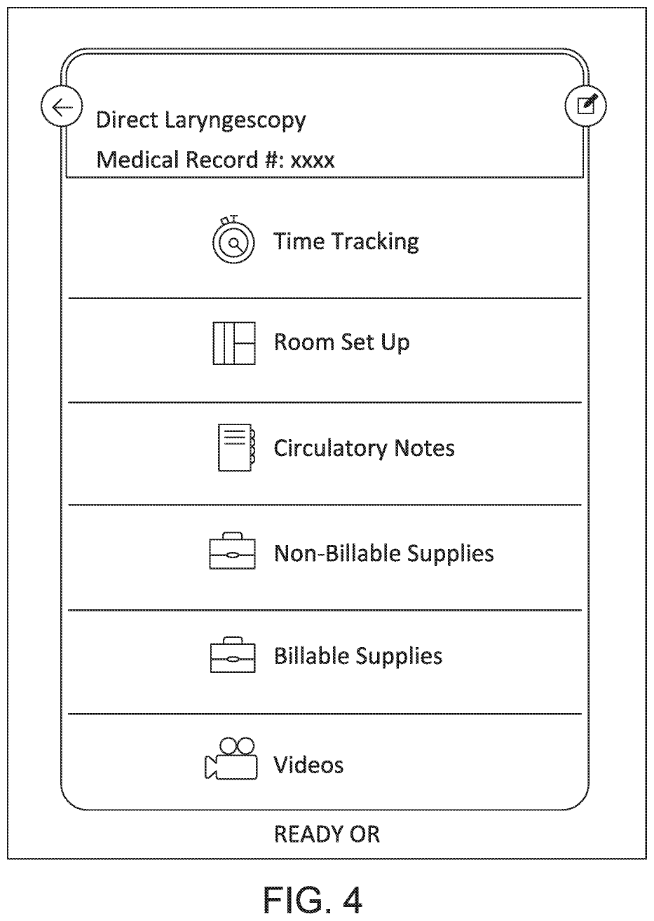

With respect to FIG. 4, each surgeon has a section of the software dedicated to their preferences. Each surgeon can have a time tracking feature, a room set up, circulator notes, billable supplies, non-billable supplies, as well as videos.

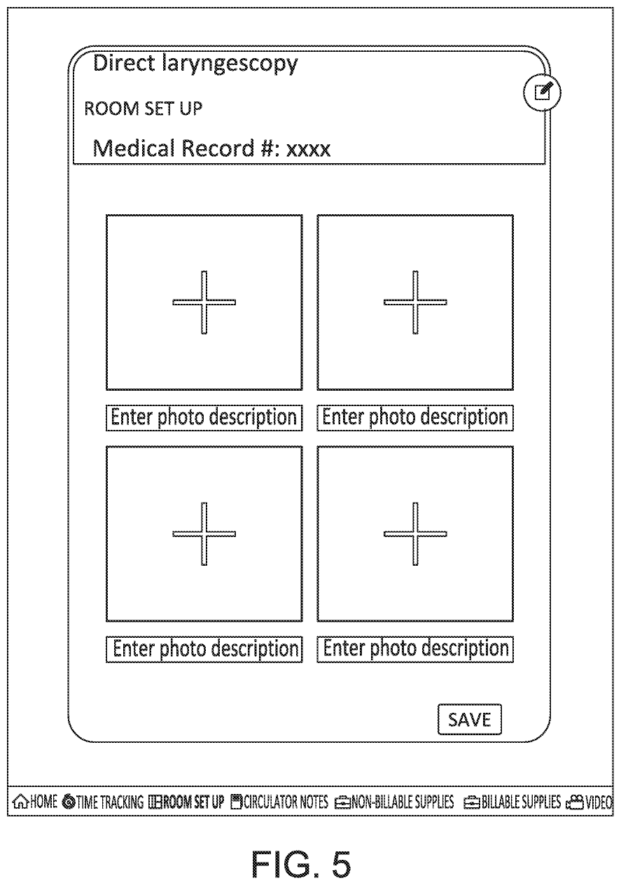

With respect to FIG. 5, the system has a room set up feature that visually depicts the surgeon's preference for a room set up, and which equipment is to be used. One issue that currently exists is that the terminology for the piece of equipment can be different between the surgeons, or the surgeons and the staff. The visual representation of the room and the equipment will make it clear which piece of equipment is desired. The surgeon could have a different room set up for each surgery, and each surgeon could have a different set up. Each room set up will have a picture of the various sections of the room, so that it will be clear to staff exactly how the room is to be set up for each surgeon, for each surgery.

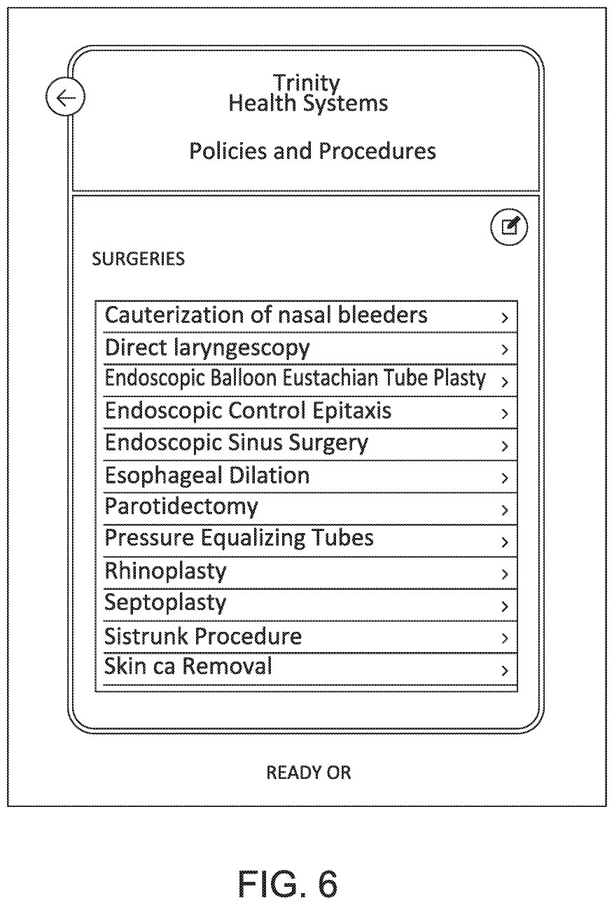

With respect to FIG. 6, each surgeon will have a list of the surgeries they perform, and the particular room set ups for each particular surgery will be listed under that surgery, under that surgeon within the software. The user of the system can find the surgeon from a directory, or can do a search by name, and when the surgeon is found, the list of available surgeries will populate. The user can choose the relevant surgery, which will then provide the surgeon's preference for that particular surgery.

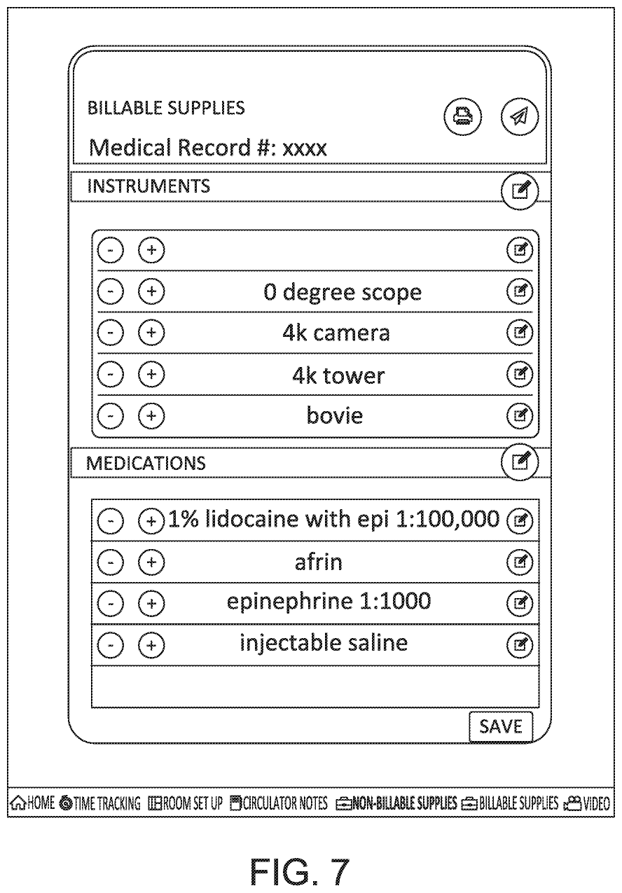

With respect to FIG. 7, the system can also have a non-billable section which creates a list of instruments and/or medications that will be used during the surgery that are non-billable. The amounts and content of each can be adjusted readily within the software.

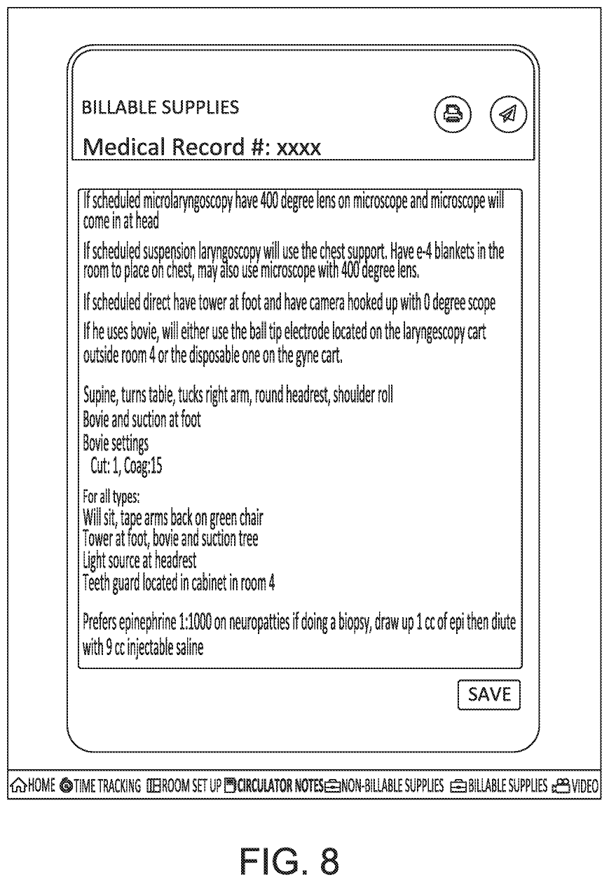

With respect to FIG. 8, the system can have a circulator notes section. This section allows the individual who helps run the operating room before, during, and after the surgery to make notes within the software for particular surgeons and/or particular surgeries. These notes can be edited and saved at any time within the software. The surgeon can also enter notes here, if the surgeon desires any changes to their normal preference, or if there is something out of the ordinary for this particular patient or procedure.

The present teachings have been described, hereinabove. It will be apparent to those skilled in the art that the above methods and apparatuses may incorporate changes and modifications without departing from the general scope of the present teachings. It is intended to include all such modifications and alterations insofar as they come within the scope of the appended claims or the equivalents thereof. Although the description above contains much specificity, this should not be construed as limiting the scope of the present teachings, but as merely providing illustrations of some of the aspects of the present teachings. Various other aspects and ramifications are possible within its scope.

Furthermore, notwithstanding that the numerical ranges and parameters setting forth the broad scope of the present teachings are approximations, the numerical values set forth in the specific examples are reported as precisely as possible. Any numerical value, however, inherently contain certain errors necessarily resulting from the standard deviation found in their respective testing measurements.

Clause 1—A system for ensuring proper set-up of an operating room and proper use of surgical tools during surgery, the system comprising a handheld electronic device, an electronic database, wherein the electronic database either resides on the handheld electronic database or the handheld electronic database has remote access to the electronic database, an electronic list of physicians stored in the electronic database, at least one electronic image of a preferred operating room set-up for each of the physicians for each of the physician's surgical procedures, wherein the at least one electronic image of the preferred operating room set up shows each piece of surgical equipment for each surgical procedure and a preferred location for each piece of surgical equipment for each surgical procedure, wherein the at least one electronic image is stored in the electronic database, an electronically selectable list of billable supplies, wherein the electronically selectable list of billable supplies is stored on the electronic database, wherein the electronic list of billable supplies is automatically transmittable to an associated patient and/or an associated insurance company, at least one instructional video, wherein the at least one instructional video contains instructions on use of the surgical equipment, wherein the instructional video contains instructions on how to trouble shoot the surgical equipment, wherein the at least one instructional video is stored on the electronic database, and a time tracker for tracking stages of an operation, wherein the time tracker is stored on the electronic database.

Clause 2—The system of clause 1, wherein the system further comprises electronic circulator notes.

Clause 3—The system of clauses 1 or 2, wherein the time tracker comprises entry time when the associated patient enter the operating room, surgery starting time, surgery ending time, and time when the associated patient is moved to a recovery room.

Clause 4—A system for ensuring proper set-up of an operating room and proper use of surgical tools during surgery, the system comprising a handheld electronic device, an electronic database, wherein the electronic database either resides on the handheld electronic database or the handheld electronic database has remote access to the electronic database, an electronic list of physicians stored in the electronic database, and at least one electronic image of a preferred operating room set-up for each of the physicians for each of the physician's surgical procedures, wherein the at least one electronic image of the preferred operating room set up shows each piece of surgical equipment for each surgical procedure and a preferred location for each piece of surgical equipment for each surgical procedure, wherein the at least one electronic image is stored in the electronic database.

Clause 5—The system of clause 4, wherein the system further comprises an electronically selectable list of billable supplies, wherein the electronically selectable list of billable supplies is stored on the electronic database, wherein the electronic list of billable supplies is automatically transmittable to an associated patient and/or an associated insurance company.

Clause 6—The system of clauses 4 or 5, wherein the system further comprises at least one instructional video, wherein the at least one instructional video contains instructions on use of the surgical equipment, wherein the instructional video contains instructions on how to trouble shoot the surgical equipment, wherein the at least one instructional video is stored on the electronic database.

Clause 7—The system of clauses 4-6, wherein the system further comprises a time tracker for tracking stages of a surgical procedure, wherein the time tracker is stored on the electronic database.

Clause 8—The system of clauses 6 or 7, wherein the system further comprises multiple participant real time video functionality, wherein the multiple participant real time video functionality is stored on the electronic database.

Clause 9—The system of clauses 4-8, wherein the system further comprises electronic circulator notes.

Clause 10—The system of clauses 7-9, wherein the time tracker comprises entry time when the associated patient enter the operating room, surgery starting time, surgery ending time, and time when the associated patient is moved to a recovery room.

Clause 11—A method for ensuring proper set-up of an operating room and proper use of surgical tools during surgery, the method comprising the steps of providing a handheld electronic device, providing an electronic database, wherein the electronic database either resides on the handheld electronic database or the handheld electronic database has remote access to the electronic database, storing an electronic list of physicians on the electronic database, and storing at least one electronic image of a preferred operating room set-up for each of the physicians for each of the physician's surgical procedures, wherein the at least one electronic image of the preferred operating room set up shows each piece of surgical equipment for each surgical procedure and a preferred location for each piece of surgical equipment for each surgical procedure, wherein the at least one electronic image is stored in the electronic database.

Clause 12—The method of clause 11, wherein the method further comprises the steps of electronically selecting a list of billable supplies, wherein the electronically selectable list of billable supplies is stored on the electronic database; creating an invoice based on billable supplies used during the surgical procedure; and automatically transmitting the invoice to an associated patient and/or an associated insurance company.

Clause 13—The method of clauses 11 or 12, wherein the method further comprises the steps of creating at least one instructional video, wherein the at least one instructional video contains instructions on use of the surgical equipment, wherein the instructional video contains instructions on how to trouble shoot the surgical equipment and storing the at least one instructional video on the electronic database.

Clause 14—The method of clauses 11-13, wherein the method further comprises the steps of tracking time for stages of the surgical procedure; and storing tracked time on the electronic database.

Clause 15—The method of clauses 11-14, wherein the method further comprises the step of providing multiple participant real time video functionality, wherein the multiple participant real time video functionality is stored on the electronic database.

Clause 16—The method of clauses 11-15, wherein the method further comprises the step of providing electronic circulator notes.

Clause 17—The method of clauses 14-16, wherein the method further comprises the steps of tracking an entry time when the associated patient enter the operating room, tracking surgery starting time, tracking surgery ending time, and tracking time when the associated patient is moved to a recovery room.

What is claimed is:

1. A system for ensuring proper set-up of an operating room and proper use of surgical tools during surgery, the system comprising:
a handheld electronic device;
an electronic database, wherein the electronic database either resides on the handheld electronic database or the handheld electronic database has remote access to the electronic database;
an electronic list of physicians stored in the electronic database;
at least one electronic image of a preferred operating room set-up for each of the physicians for each of the physician's surgical procedures, wherein the at least one electronic image of the preferred operating room set up shows each piece of surgical equipment for each surgical procedure and a preferred location for each piece of surgical equipment for each surgical procedure, wherein the at least one electronic image is stored in the electronic database;
an electronically selectable list of billable supplies, wherein the electronically selectable list of billable supplies is stored on the electronic database, wherein the electronic list of billable supplies is automatically transmittable to an associated patient and/or an associated insurance company;
at least one instructional video, wherein the at least one instructional video contains instructions on use of the surgical equipment, wherein the instructional video contains instructions on how to trouble shoot the surgical equipment, wherein the at least one instructional video is stored on the electronic database; and
a time tracker for tracking stages of an operation, wherein the time tracker is stored on the electronic database.

2. The system of claim 1, wherein the system further comprises:
electronic circulator notes.

3. The system of claim 1, wherein the time tracker comprises:
entry time when the associated patient enter the operating room;
surgery starting time;
surgery ending time; and
time when the associated patient is moved to a recovery room.

4. A system for ensuring proper set-up of an operating room and proper use of surgical tools during surgery, the system comprising:
a handheld electronic device;
an electronic database, wherein the electronic database either resides on the handheld electronic database or the handheld electronic database has remote access to the electronic database;
an electronic list of physicians stored in the electronic database; and
at least one electronic image of a preferred operating room set-up for each of the physicians for each of the physician's surgical procedures, wherein the at least one electronic image of the preferred operating room set up shows each piece of surgical equipment for each surgical procedure and a preferred location for each piece of surgical equipment for each surgical procedure, wherein the at least one electronic image is stored in the electronic database.

5. The system of claim 4, wherein the system further comprises:
an electronically selectable list of billable supplies, wherein the electronically selectable list of billable supplies is stored on the electronic database, wherein the electronic list of billable supplies is automatically transmittable to an associated patient and/or an associated insurance company.

6. The system of claim 4, wherein the system further comprises:
at least one instructional video, wherein the at least one instructional video contains instructions on use of the surgical equipment, wherein the instructional video contains instructions on how to trouble shoot the surgical equipment, wherein the at least one instructional video is stored on the electronic database.

7. The system of claim 4, wherein the system further comprises:
a time tracker for tracking stages of a surgical procedure, wherein the time tracker is stored on the electronic database.

8. The system of claim 6, wherein the system further comprises:
multiple participant real time video functionality, wherein the multiple participant real time video functionality is stored on the electronic database.

9. The system of claim 4, wherein the system further comprises:
electronic circulator notes.

10. The system of claim 7, wherein the time tracker comprises:
entry time when the associated patient enter the operating room;
surgery starting time;
surgery ending time; and
time when the associated patient is moved to a recovery room.

11. A method for ensuring proper set-up of an operating room and proper use of surgical tools during surgery, the method comprising the steps of:
providing a handheld electronic device;
providing an electronic database, wherein the electronic database either resides on the handheld electronic database or the handheld electronic database has remote access to the electronic database;
storing an electronic list of physicians on the electronic database; and
storing at least one electronic image of a preferred operating room set-up for each of the physicians for each of the physician's surgical procedures, wherein the at least one electronic image of the preferred operating room set up shows each piece of surgical equipment for each surgical procedure and a preferred location for each piece of surgical equipment for each surgical procedure, wherein the at least one electronic image is stored in the electronic database.

12. The method of claim 11, wherein the method further comprises the steps of:
electronically selecting a list of billable supplies, wherein the electronically selectable list of billable supplies is stored on the electronic database;
creating an invoice based on billable supplies used during the surgical procedure; and
automatically transmitting the invoice to an associated patient and/or an associated insurance company.

13. The method of claim 11, wherein the method further comprises the steps of:
creating at least one instructional video, wherein the at least one instructional video contains instructions on use of the surgical equipment, wherein the instructional video contains instructions on how to trouble shoot the surgical equipment; and
storing the at least one instructional video on the electronic database.

14. The method of claim 11, wherein the method further comprises the steps of:
tracking time for stages of the surgical procedure; and
storing tracked time on the electronic database.

15. The method of claim 11, wherein the method further comprises the step of:
providing multiple participant real time video functionality, wherein the multiple participant real time video functionality is stored on the electronic database.

16. The method of claim 11, wherein the method further comprises the step of:
providing electronic circulator notes.

17. The method of claim 14, wherein the method further comprises the steps of:
tracking an entry time when the associated patient enter the operating room;
tracking surgery starting time;
tracking surgery ending time; and
tracking time when the associated patient is moved to a recovery room.
